(19) United States
(12) Patent Application Publication
(10) Pub. No.: US 2006/0049052 A1
(43) Pub. Date: Mar. 9, 2006

(54) Ampholytic buffer having high buffering capacity and high conductivity in isoelectric form

(75) Inventors: Gyula Vigh, Magnolia, TX (US); Sanjiv Kumar Lalwani, College Station, TX (US)

Correspondence Address: CHALKER FLORES, LLP, 2711 LBJ FRWY, Suite 1036, DALLAS, TX, 75234, US

(73) Assignee: The Texas A&M University System, College Station, TX (US)

(21) Appl. No.: 11/219,351

(22) Filed: Sep. 2, 2005

Related U.S. Application Data
(60) Provisional application No. 60/606,691, filed on Sep. 2, 2004.

(51) Int. Cl. C07K 1/26; G01N 27/447

(52) U.S. Cl. 204/450; 204/600

(57) ABSTRACT

Ampholytic buffers having a high buffering capacity and a high conductivity in the isoelectric state were synthesized by creating molecules in which four or more bonds separate the charge-carrying or chargeable atoms of the pI-determining weak electrolyte functional groups and, simultaneously, the charge-carrying or chargeable atom of the weak or strong electrolyte charge-balancing functional group.

This application claims claims priority to U.S. Provisional Application Ser. No. 60/606,691, filed Sep. 2, 2004, the contents of which are incorporated by reference herein in their entireties.

### TECHNICAL FIELD OF THE INVENTION

This invention relates to ampholytic buffers that have high buffering capacities and high conductivities in their isoelectric forms and their methods of preparation. The primary application areas of the ampholytic buffers that have high buffering capacities and high conductivities in their isoelectric forms are in the analytical and preparative-scale isoelectric focusing and isoelectric trapping separations of ampholytic components.

### BACKGROUND OF THE INVENTION

Without limiting the scope of the invention, its background is described in connection with isolectric buffers. Electrophoretic protein separations, and particularly isoelectric focusing (IEF) and isoelectric trapping (IET) separations [1,2] are gaining acceptance as viable alternatives to chromatographic separation methods [3]. In IEF, ampholytic species are separated, in an applied electric field, in a pH gradient, based on the differences in their isoelectric points (pI values) [4]. In IET, accomplished in multicompartmental electrolyzers (MCEs) [5-9], ampholytic analytes are isolated into separation compartments that are formed by buffering membranes whose pH values bracket the pI values of the ampholyte of interest. Typically, in IET [5-9], sample solutions containing mixtures of proteins are recirculated through the separation compartments, external coolers and reservoirs. The electric field, orthogonal to the direction of the flow, moves the proteins through the buffering membranes into separation compartments formed by buffering membranes whose pH values bracket the pI of the target protein. The advantage of classical IET [5-9] is that proteins can be isolated in their pure, isoelectric form between the buffering membranes made, for example, by copolymerizing acrylamide, N,N′-methylenebisacrylamide, and sufficient amounts of the appropriate acrylamido weak acid or weak base derivatives (the buffering species) and an acrylamido strong acid or strong base derivative (the titrating species) [10, 11], commercially available as Immobilines [12]. These Immobiline-based buffering membranes have been successfully used in a diverse set of applications (see, e.g. [13-26]). Recently, the Gradiflow electrophoretic binary protein separation unit, the BF200 [27] has been modified to operate in IET mode [17, 22].

Since the solubility of proteins in their pure, isoelectric form is significantly lower than in their charged form [3, 9, 12], conventional IET often suffers from protein precipitation when the protein load is high, limiting the productivity of the process. In order to eliminate this problem, pH-biased IET was invented and implemented in a modified BF200IET unit [26]. In pH-biased IET, proteins are isolated into solutions of isoelectric buffers (ampholytic buffers in their isoelectric state) that establish a solution pH different from the pI of the target protein, keeping the protein in a charged state and insuring adequate protein solubility [26]. These isoelectric ampholytic buffers, also known as biasers, need to be selected such that (i) their pI is bracketed by the pH values of the buffering membranes of the respective separation compartments, (ii) their pI is different from the pI of the protein(s) of interest, and (iii) they have good buffering capacity in their isoelectric state [4]. Currently, very few compounds are commercially available that meet these criteria, which seriously limits the flexibility and utility of pH-biased IET.

Fullarton and Kenny [28], Hjertén et al. [29] and Righetti et al. [30-33] published electrophoretic separations, and particularly capillary electrophoretic separations, that were obtained using isoelectric ampholytic buffers that had closely spaced pKa values. The authors explained that these separations were efficient because these ampholytic buffers had low conductivities in their isoelectric states (at their pI values) and thus permitted the use of high electric field strengths.

In a U.S. patent Hjerten et al. [29] disclosed that ampholytic buffers that have high buffering capacities and low conductivities in their isoelectric states (termed isoelectric buffers) can be synthesized (i) by incorporating a weakly acidic and a weakly basic functional group into a molecule provided that the absolute value of the difference between the pI value of the molecule and its closest pK value is less than 1.5 (i.e., |pI-pKI|<1.5), (ii) by incorporating two weakly acidic and one weakly basic or permanently cationic functional group into a molecule, and also, by incorporating two weakly basic and one weakly acidic or permanently anionic functional group into a molecule, provided that the absolute value of the difference between the pI value of the molecule and its closest pK value is less than 1.5 (i.e., |pI-pK|<1.5), and (iii) by either of method (i) or (ii), provided, additionally, that the molecule has a large relative molecular mass, e.g., the molecule contains a long polymeric chain.

Though an ampholytic buffer whose conductivity is low in the isoelectric state, as promoted by Fullarton and Kenny [28], Hjerten et al. [29] and Righetti et al. [30-33], might be advantageous in capillary electrophoretic separations, low conductivity is detrimental when such an isoelectric buffer is used in the receiving stream of an IET system because, due to the low conductivity of the receiving stream, a large portion of the potential applied across the anode and cathode of the IET system drops over the receiving stream where it does not contribute to the IET separation. This leaves less of the applied potential to drop over the feed stream and the buffering membrane where the actual separation occurs, which reduces the efficacy of the IET process. Thus, there is a great need for ampholytic buffers that possess high buffering capacities and high conductivities in their isoelectric state (around their pI values), buffers that can act as efficient biasers in pH-biased IET.

The present inventors now have developed ampholytic buffers that possess high buffering capacities and high conductivities in their isoelectric state and can act as efficient biasers in pH-biased IET.

### SUMMARY OF THE INVENTION

In a first aspect, the present invention provides an ampholytic buffer having a unique pI value and a high buffering capacity and a high conductivity in isoelectric state, that includes a molecule with a first weakly acidic functional group with a first pKa value, a second weakly acidic functional group with a second pKa value, and a weakly basic or a cationic functional group wherein the first weakly acidic functional group and the second weakly acidic functional group are similar or different in structure, the pI value of the ampholytic buffer lies between the pKa values of the weakly acidic functional groups, the absolute value of the difference between the pKa values of the first and the second weakly acidic functional groups is less than 3, the charge-carrying or chargable atoms of the first and the second weakly acidic functional groups are four or more covalent bonds away from each other and, simultaneously, the charge-carrying or chargable atoms of both of the first and the second weakly acidic functional groups are four or more covalent bonds away from the charge-carrying or chargable atom of the weakly basic or cationic functional group.

The cationic functional group of the isoelectric buffer is a quaternary ammonium group, because it is less prone to N-oxide formation than the ampholytic buffers that contain primary, secondary or tertiary amines. This is an especially valuable feature when the ampholytic buffers of the present invention are used in the anode compartment or anodic separation compartment of the IET apparatus.

In a second aspect, the present invention provides an ampholytic buffer having a unique pI value and a high buffering capacity and a high conductivity in isoelectric state, including a molecule with a first weakly basic functional group with a first pKa value for its corresponding conjugate acid form, a second weakly basic functional group with a second pKa value for its corresponding conjugate acid form, and a weakly acidic or an anionic functional group wherein the first weakly basic functional group and the second weakly basic functional group are similar or different in structure, the pI value of the ampholytic buffer lies between the pKa values of the conjugate acid forms of the weakly basic functional groups, the absolute value of the difference between the pKa values of the conjugate acid forms of the first and the second weakly basic functional groups is less than 3, the charge-carrying or chargable atoms of the first and the second weakly basic functional groups are four or more covalent bonds away from each other and, simultaneously, the charge-carrying or chargable atoms of both of the first and the second weakly basic functional groups are four or more covalent bonds away from the charge-carrying or chargable atom of the weakly acidic or anionic functional group.

In a third aspect, the present invention provides an ampholytic buffer having a unique pI value and a high buffering capacity and a high conductivity in isoelectric state, that includes a molecule with a weakly acidic functional group with a pKa value characteristic of the weakly acidic functional group and a weakly basic functional group with a pKa value characteristic of its corresponding conjugate acid form, wherein the pI value of the isoelectric buffer lies between the pKa value of the weakly acidic functional group and the pKa value of the conjugate acid form of the weakly basic functional group, the absolute value of the difference between the pKa value of the weakly acidic functional group and the pKa value of the conjugate acid form of the weakly basic functional group is less than 3, the charge-carrying or chargable atoms of the weakly acidic and weakly basic functional groups are four or more covalent bonds away from each other.

In a fourth aspect, the present invention provides a method for forming an ampholytic buffer of the first aspect by: reacting a secondary amine with a first ester formed from a first weak acid, the first weak acid having a pKa value between 1 and 14, the first weak acid having a functional group capable of reacting with the secondary amine, the reaction resulting in an ester-group bearing tertiary amine; reacting the ester-group bearing tertiary amine with a second ester formed from a second weak acid, the second weak acid having a pKa value between 1 and 14, the second weak acid having a functional group capable of reacting with the ester-group bearing tertiary amine, the reaction resulting in a quaternary ammonium compound bearing two ester groups, the first weak acid and the second weak acid having a similar or a different structure, the first ester and the second ester having a similar or a different structure; hydrolyzing the first ester and the second ester group of the quaternary ammonium diester compound forming an ampholytic compound having, simultaneously, four or more bonds between the charged or chargeable atoms of the first weakly acidic functional group, the second weakly acidic functional group and the nitrogen atom of the quaternary ammonium group; and recovering, in a pure isoelectric form, the ampholytic buffer.

In a fifth aspect, the present invention provides a method for forming an ampholytic buffer of the first aspect that includes: reacting a primary amine with a first ester formed from a first weak acid, the first weak acid having a pKa value between 1 and 14, the first weak acid having a functional group capable of reacting with the primary amine, the reaction resulting in an ester-group bearing secondary amine; reacting the ester-group bearing secondary amine with a second ester formed from a second weak acid, the second weak acid having a pKa value between 1 and 14, the second weak acid having a functional group capable of reacting with the ester-group bearing secondary amine, the reaction resulting in a tertiary amine beraing two ester groups, the first weak acid and the second weak acid having a similar or a different structure, the first ester and the second ester having a similar or different structure; hydrolyzing the first ester and the second ester group of the tertiary amine diester compound forming an ampholytic compound having, simultaneously, four or more bonds between the charged or chargeable atoms of the first weakly acidic functional group, the second weakly acidic functional group and the nitrogen atom of the tertiary amino group; and recovering, in a pure isoelectric form, the ampholytic buffer.

In a sixth aspect, the present invention provides a method for forming an ampholytic buffer of the first aspect by: reacting a primary amine with a first ester formed from a first weak acid, the first weak acid having a pKa value between 1 and 14, the first weak acid having a functional group capable of reacting with the primary amine, the reaction resulting in an ester-group bearing secondary amine; reacting the ester-group bearing secondary amine with a second ester formed from a second weak acid, the second weak acid having a pKa value between 1 and 14, the second weak acid having a functional group capable of reacting with the ester-group bearing secondary amine, the reaction resulting in a tertiary amine bearing two ester groups, the first weak acid and the second weak acid having a similar or a different structure, the first ester and the second ester heaving a similar or different structure; reacting the diester-bearing tertiary amine with a reagent having a functional group capable of reacting with the tertiary amine bearing two ester groups, the reaction resulting in a quaternary ammonium compound bearing two ester groups; hydrolyzing the first ester and the second ester group of the quaternary ammonium diester compound forming an ampholytic compound having, simultaneously, four or more bonds between the charged or chargeable atoms of the first weakly acidic functional group, the second weakly acidic functional group and the nitrogen atom of the quaternary ammonium group; and recovering, in a pure isoelectric form, the ampholytic buffer.

In a seventh aspect, the present invention provides a method for forming an ampholytic buffer of the first aspect by: reacting a primary amine with a first ester formed from a first weak acid, the first weak acid having a pKa value between 1 and 14, the first weak acid having a functional group capable of reacting with the primary amine, the reaction resulting in a secondary amine bearing an ester group; reacting the secondary amine bearing the ester group with a reagent having a functional group capable of reacting with the ester-bearing secondary amine, the reaction resulting in a tertiary amine bearing an ester group; reacting the ester-bearing tertiary amine with a second ester formed from a second weak acid, the second weak acid having a pKa value between 1 and 14, the second weak acid having a functional group capable of reacting with the ester-group bearing tertiary amine, the reaction resulting in a quaternary ammonium compound bearing two ester groups, the first weak acid and the second weak acid having a similar or a different structure, the first ester and the second ester having a similar or different structure; hydrolyzing the first ester and the second ester group of the quaternary ammonium diester compound forming an ampholytic compound having, simultaneously, four or more bonds between the charged or chargeable atoms of the first weakly acidic group, the second weakly acidic group and the nitrogen atom of the quaternary ammonium group; and recovering, in a pure isoelectric form, the ampholytic buffer.

In an eighth aspect, the present invention provides a method for forming an ampholytic buffer of the second aspect by: selecting a diamino alcohol having a first weakly basic functional group and a second weakly basic functional group and a hydroxyl group, the first weakly basic functional group having a conjugate acid form with a pKa value between 1 and 14, the second weakly basic functional group having a conjugate acid form with a pKa value between 1 and 14, the first and the second weakly basic functional groups having a similar or a different structure; converting the hydroxyl group of the diamino alcohol into a weakly acidic functional group or an anionic functional group forming an ampholytic compound having, simultaneously, four or more bonds between the charged or chargeable atoms of the first weakly basic functional group, the second weakly basic functional group and the weakly acidic or anionic functional group; and recovering, in a pure isoelectric form, the ampholytic buffer.

In a ninth aspect, the present invention includes a method for forming an ampholytic buffer of the second aspect that includes the steps of: selecting a first secondary amine having a conjugate acid form with a pKa value between 1 and 14; selecting a second secondary amine having a conjugate acid form with a pKₐ value between 1 and 14, the first and the second secondary amines having a similar or different structure; selecting a difunctional reagent having a first reactive group, a second reactive group and a hydroxyl group or a protected hydroxyl group, the first and the second reactive groups having a similar or different structure; reacting, simultaneously or sequentially, the first secondary amine with the first reactive group of the difunctional reagent and the second secondary amine with the second reactive group of the difunctional reagent forming a diamino compound and preserving, intact, the hydroxyl group or protected hydroxyl group; converting the hydroxyl group or the protected hydroxyl group of the diamino compound into a weakly acidic functional group or an anionic functional group forming an ampholytic compound having, simultaneously, four or more bonds between the charged or chargeable atoms of the first weakly basic functional group, the second weakly basic functional group and the weakly acidic or anionic functional group; and recovering, in a pure isoelectric form, the ampholytic buffer.

In a tenth aspect, the present invention provides a method for the use of the ampholytic isoelectric buffers according to the first, second or third aspect of the present invention in electrophoretic separations.

In an eleventh aspect, the present invention provides a method for the use of the ampholytic isoelectric buffers according to the first, second or third aspect of the present invention in isoelectric focusing separations.

In a twelfth aspect, the present invention provides a method for the use of the isoelectric buffers according to the first, second or third aspect of the present invention in isoelectric trapping separations.

### BRIEF DESCRIPTION OF THE DRAWINGS

For a more complete understanding of the features and advantages of the present invention, reference is now made to the detailed description of the invention along with the accompanying figures, and in which:

FIG. 1 is a generic structure of the ampholytic isoelectric buffers according to the first aspect of the present invention. R and R′ is selected from the group that includes alkyl, aryl, alkylaryl, oxyalkyl, oxyaryl and oxyalkylaryl groups, same or different, R″ and R′″ is selected from the group that includes alkyl, aryl, alkylaryl, oxyalkyl, oxyaryl and oxyalkylaryl groups having two or more carbon atoms, same or different;

FIG. 2 is an example of the reaction scheme for the synthesis of isoelectric buffers according to the first aspect of the present invention;

FIG. 3 is a generic structure of some of the amine-based isoelectric buffers according to the second and third aspects of the present invention;

FIG. 4 is a general scheme for the synthesis of some of the amine-based isoelectric buffers according to the present invention;

FIG. 5 is a general scheme for the synthesis of some of the amine-based isoelectric buffers according to the present invention;

FIG. 6 is a picture of the ¹H-NMR (top panel) and ¹³C-NMR (bottom panel) spectra of BCPDEAH;

FIG. 7 is a picture of the ¹H-¹H COSY spectrum for BCPDEAH and the corresponding signal assignments;

FIG. 8 is a picture of the ¹H-¹³C HETCOR spectrum for BCPDEAH and the corresponding signal assignments;

FIG. 9 is a picture of the ESI-MS of BCPDEAH in positive ion (top) and negative ion (bottom) modes;

FIG. 10 is a picture of the ¹H- (top panel) and ¹³C-NMR spectra (bottom panel) for BDASP with the corresponding signal assignments;

FIG. 11 is a picture of the ¹H-¹H COSY spectrum for BDASP with the corresponding signal assignments;

FIG. 12 is a picture of the ¹H-¹³C HETCOR spectrum for BDASP with the corresponding signal assignments;

FIG. 13 is a picture of the ESI-MS of BDASP in the positive ion (top) and negative ion (bottom) modes;

FIG. 14 is a ball-and-stick image of the X-ray crystal structure of BDASP;

FIG. 15 is a picture of the ¹H- (top) and ¹³C-NMR (bottom) spectra of DMSP;

FIG. 16 is a picture of the ¹H-¹H COSY spectrum of DMSP with the corresponding signal assignments;

FIG. 17 is a picture of the ¹H-¹³C HETCOR spectrum of DMSP with the corresponding signal assignments;

FIG. 18 is a picture of the ESI-MS of DMSP in positive ion (top) and negative ion (bottom) modes;

FIG. 19 is a ball-and-stick image of the single crystal X-ray structure of DMSP;

FIG. 20 is a picture of the ¹H- (top) and ¹³C- (bottom) NMR spectra for BDPSP with the corresponding signal assignments;

FIG. 21 is a picture of the ¹H-¹H COSY spectrum for BDPSP with the corresponding signal assignments;

FIG. 22 is a picture of the ¹H-¹³C HETCOR spectrum for BDPSP with the corresponding signal assignments;

FIG. 23 is a picture of the ESI-MS spectra for BDPSP in positive (top) and negative ion (bottom) modes;

FIG. 24 is a ball-and-stick image of the single crystal X-ray structure of BDPSP;

FIG. 25 is a picture of the ¹H- (top) and ¹³C- (bottom) NMR spectra for DPSP with the corresponding signal assignments;

FIG. 26 is a picture of the ¹H-¹H COSY spectrum for DPSP with the corresponding signal assignments;

FIG. 27 is a picture of the ¹H-¹³C HETCOR spectrum for DPSP and the corresponding signal assignments; and

FIG. 28 is a picture of the ESI-MS spectra for DPSP in the positive (top) and negative (bottom) ion modes.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

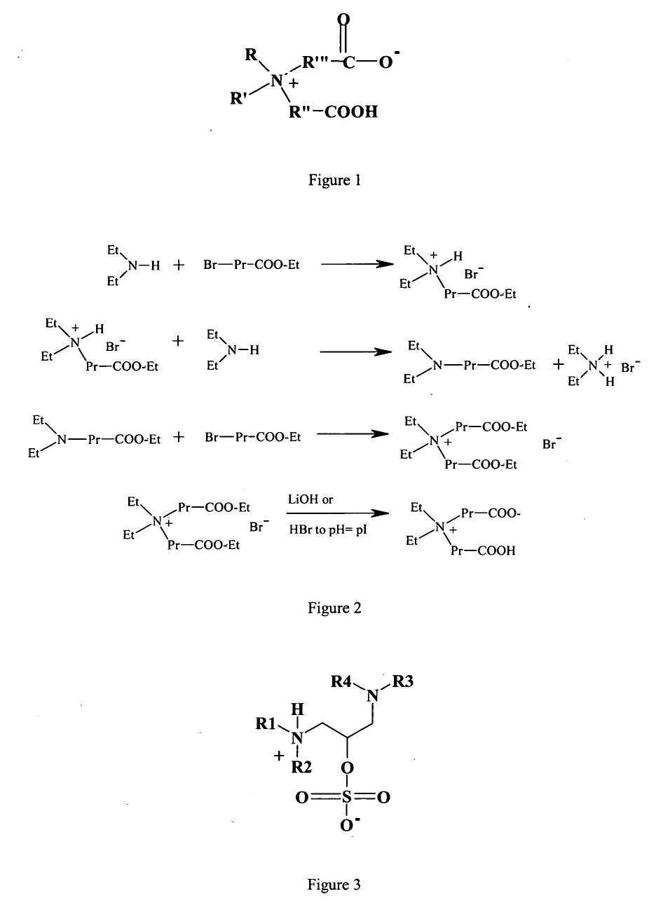

FIG. 1 shows the generic structure of the ampholytic isoelectric buffers that contain two carboxylic acid groups and an N-oxidation-resistant quaternary ammonium group according to the first aspect of the present invention.

A family of carboxylic acid-based ampholytic isoelectric buffers can be synthesized according to the synthesis scheme shown as an example in FIG. 2. First, 2 equivalents of a secondary amine are reacted with 1 equivalent of an alkylbromoalkanoate. As 1 equivalent of the secondary amine attaches to the alkylalkanoate chain forming a protonated, tertiary ammonium intermediate, the second equivalent of the secondary amine deprotonates the tertiary ammonium intermediate (pKₐ of the conjugate acid of the secondary amine is slightly higher than the pKₐ of the conjugate acid of the tertiary amine intermediate). As a result, the bromide salt of the protonated secondary amine precipitates out from the reaction mixture and can be separated from the tertiary amine intermediate by, e.g., filtration. Next, 1 equivalent of the tertiary amine intermediate is reacted with 1 equivalent of an alkyl bromoalkanoate (either the same as the first one or a different one) to form the bromide salt of the quaternary ammonium intermediate. Finally, the ester functionalities are hydrolyzed (with either an aqueous strong acid or strong base) to form the free carboxylic acids. The product can then be obtained in isoelectric form by crystallization from a concentrated aqueous solution titrated to the pH equal to the pI of the ampholytic buffer or by desalting the concentrated aqueous solution in an IET system. Since a large variety of secondary amines and halocarboxylic acids are available, this synthesis scheme has great flexibility in terms of the derivatives that can be synthesized and their pI values.

FIG. 3 shows a generic structure for some of the amine-based isoelectric buffers according to the second and third aspects of the present invention (containing two amine groups and one sulfate group).

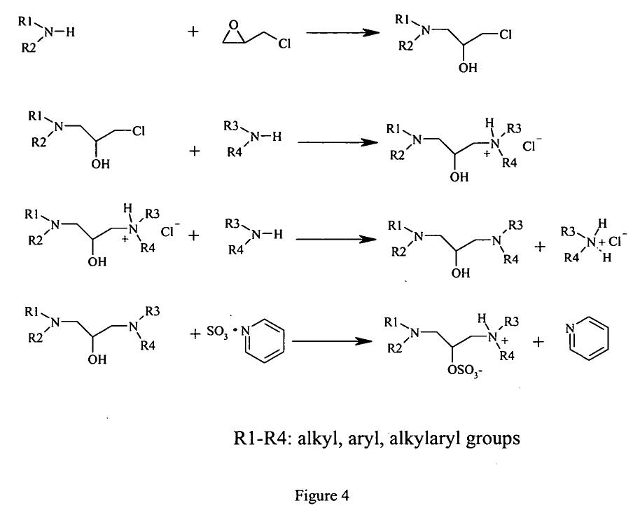

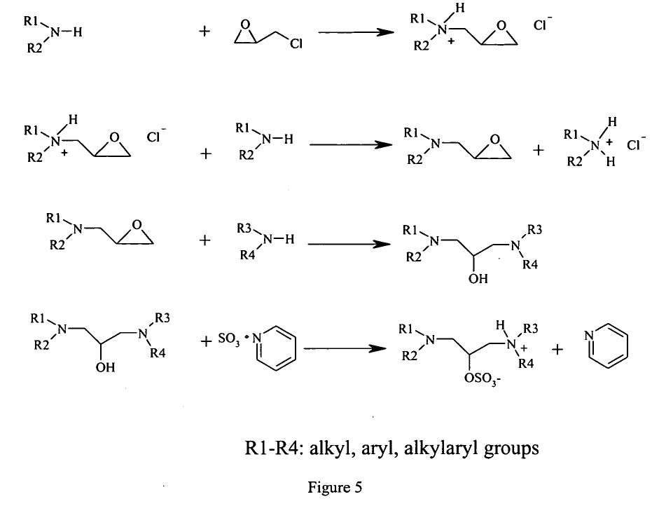

FIGS. 4 and 5 illustrate schemes for the synthesis of some of the amine-based isoelectric buffers according to the second aspect of the present invention. According to FIG. 4, 1 equivalent of a secondary amine is reacted with 1 equivalent of an epihalohydrin, such as epichlorohydrin, in tetrahydrofuran as solvent forming an amino alcohol intermediate. Next, 1 equivalent of the amino alcohol intermediate is reacted with 1 equivalent of a secondary amine (either the same as the first one or a different one) forming a diamino alcohol intermediate, while another 1 equivalent of the secondary amine deprotonates the diaminoalcohol intermediate (pKₐ of the conjugate acid form of the secondary amine is slightly higher than the pKₐ of the conjugate acid form of the diamino alcohol intermediate). The halide salt (e.g., the chloride salt) of the protonated secondary amine precipitates out from the reaction mixture and can be separated from the diamino alcohol intermediate by, e.g., filtration. Finally, the diamino alcohol intermediate can be sulfated with SO₃XPyr to form the ampholytic buffer in the salt form. The product can then be obtained in isoelectric form by crystallization from a concentrated aqueous solution titrated to the pH equal to the pI of the isoelectric buffer, or by isoelectric trapping in an IET apparatus.

### EXAMPLE 1

Synthesis of N,N-bis(carboxypropyl)diethyl ammonium hydroxide, inner salt (BCPDEAH): BCPDEAH was synthesized as follows. First, 10.37 g (0.142 mol) diethylamine (DEA) was mixed with 13.5 g (0.069 mol) ethyl 4-bromobutyrate (EBB) in a 250 mL round bottom flask fitted with an ice-water cooled condenser. The flask was warmed in a heating mantle with the rheostat set at 45%. Heavy solid formation was observed within the first 30 minutes of heating. After 3 hours of heating, the reaction mixture was cooled to room temperature. 500 mL of acetone was added and the mixture was stirred into a slurry that was filtered and the solids (diethylammonium bromide) were washed with 25 mL of acetone, three times. Acetone and unreacted DEA were removed from the mixture under reduced pressure. Upon cooling the mixture, additional white solids (diethylammonium bromide) were formed which were also filtered-off. The structure of the tertiary amine intermediate was confirmed by ¹H- and ¹³C-NMR spectroscopy.

The whole batch of the tertiary amine intermediate from the previous step was mixed with 13.5 g (0.069 mol) EBB in a 250 mL round bottom flask fitted with an ice-water cooled condenser. The flask was warmed in a heating mantle with the rheostat set at 45%. After 10.5 hours, the reaction mixture was cooled to room temperature. 200 mL of deionized water were added and the mixture was stirred into a homogenous solution, which was extracted by tert-butyl methyl ether to selectively remove the contaminants from the aqueous phase. Then, 11.2 g (0.28 mol) NaOH were dissolved in 100 mL deionized water and mixed into the processed aqueous solution of the quaternary ammonium intermediate. The reaction mixture was left stirring at room temperature. Complete hydrolysis was seen after 14 hours. Ethanol formed from the hydrolysis was removed under reduced pressure.

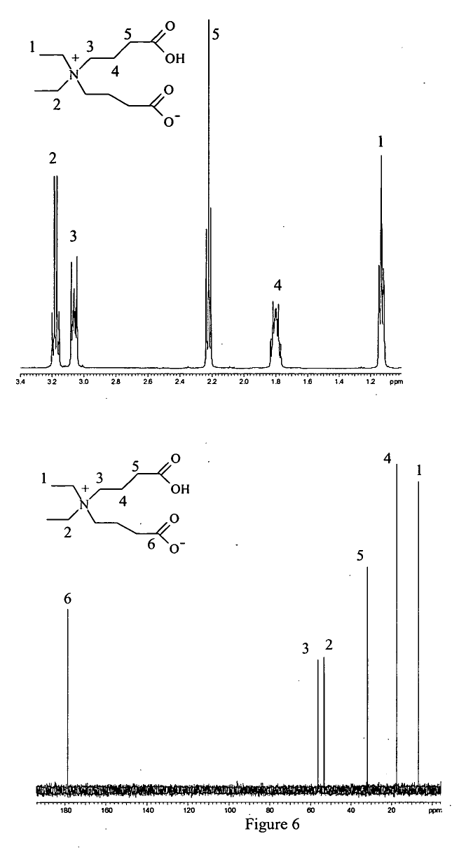

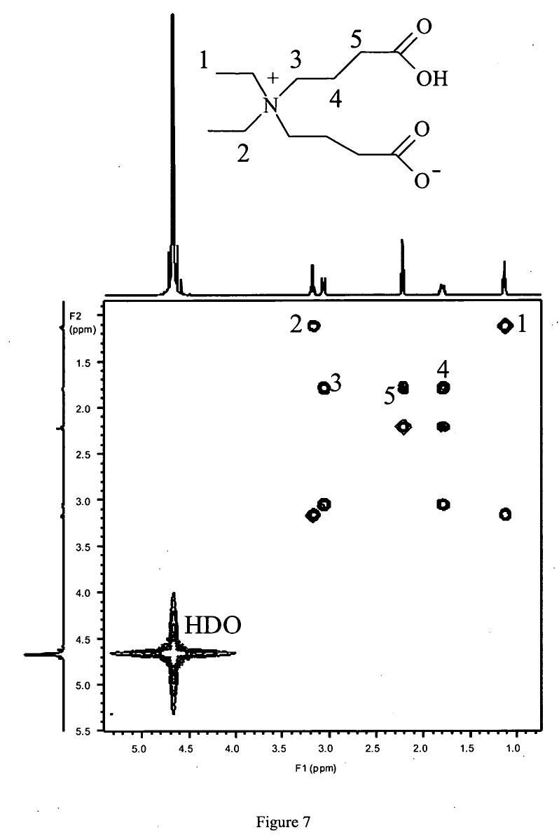

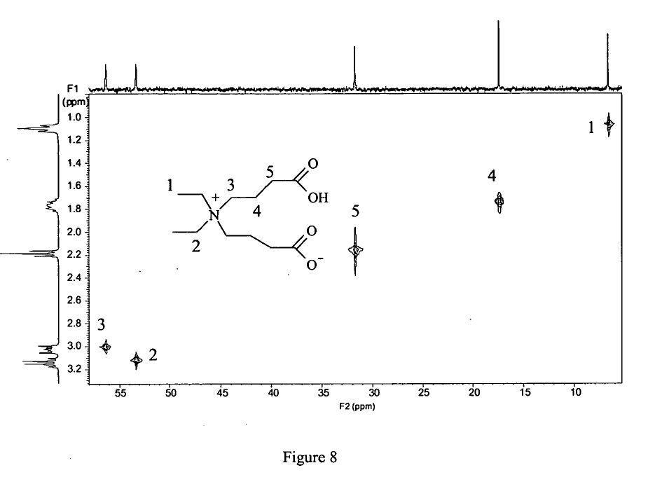

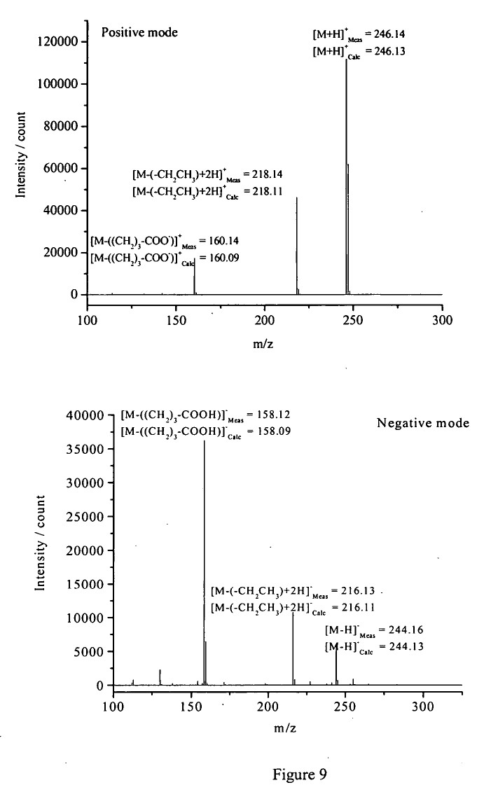

The aqueous solution was desalted and the ampholytic buffer brought to isoelectric state in an IET apparatus. The pH of the concentrated aqueous solution of isoelectric BCPDEAH was 3.8, equal to its pI value. After liophylization, the solid product was characterized by ¹H- and ¹³C-NMR. FIG. 6 shows the ¹H- and the ¹³C-NMR spectra, FIGS. 7 and 8 show the results of the ¹H-¹H COSY and ¹H-¹³C HETCOR NMR experiments. The identity and purity of the final product were further confirmed by high resolution ESI-MS. The ESI-MS spectra in positive and negative ion modes are shown in FIG. 9. Only signals corresponding to BCPDEAH and its fragment ions are seen.

### EXAMPLE 2

Synthesis of 1,3-bis(N,N-dimethylamino)-2-O-sulfo-propane (BDASP): BDASP was synthesized via sulfation of the alcohol group of 1,3-bis(N,N-dimethylamino)-2-propanol (BDAP) using sulfur trioxide pyridine complex (SO₃XPyr).

Fit a clean, 250 mL, three-neck round bottom flask with an ice-water cooled condenser. Add 40 mL DMF to the flask, set the flask in an oil bath. Add 30.0 g (0.205 mol) BDAP into the flask and heat it to 65° C. while stirring the solution with a half-inch football-shaped magnetic stir bar on a stir plate. To the warm, stirring solution add 35.93 g (0.227 mol) SO₃XPyr and continuously heat and stir the mixture at 65° C. for 4 hours. Take the flask off of the oil bath and set it to cool on a cork O-ring with paper towels underneath (to absorb the oil).

To the cooled reaction mixture, add 250 mL DMF and with the aid of a spatula, manually dislodge the solids and remove the chunks from the round bottom flask into a 500 mL beaker. Add 450 mL DMF into the beaker and make a slurry of the solids. Filter the slurry using a Buchner funnel and a suction flask. Remove the solid cake and filter paper from the funnel, and in another 500 mL beaker make a slurry with 450 mL acetone. Filter the slurry, make another slurry of the solids in 450 mL ethanol, and filter again. Let the solids dry inside the fume hood.

Dissolve the solids in minimum volume (around 200 mL) of deionized water. Measure the pH of the solution and titrate it to pH=8.0 using a 5M LiOH solution (if the solution pH is below 8.0) or 2M sulfuric acid (H₂SO₄) (if the solution pH is above 8.0). Remove about 100 mL of water using a rotovap with the water bath set at 65° C. Let the solution cool in the fume hood with continuous stirring for 24 hours. Filter the slurry, wash the solid cake with cold ethanol three times and let the cake dry in the fume hood. Analyze both the crystallization mother liquor and the solids by CE and repeat crystallization from deionized water if necessary.

The pI of BDASP was determined by indirect UV detection CE. Using a 26 :m I.D. bare fused silica capillary, Lₜ=26.1 cm, L_{d}=19.7 cm at 15 kV in positive to negative polarity, T=25 EC, UV detector set at 214 nm, conventional CE was carried out in 20 mM tris(hydroxymethyl) aminomethane (Tris) and 20 mM benzylamine BGEs titrated with pTSA. The approximate pI of BDASP was determined to be 8.0. Indirect UV detection CE using a 20 mM acetic acid BGE titrated with imidazole to pH=4.56, run on a 26 :m I.D. bare fused silica capillary, Lₜ=26.5 cm, L_{d}=19.7 cm at 25 kV, positive to negative polarity, T=25 EC, UV detector set at 214 nm, and a 20 mM Tris, 0.1 mM hexadecyltrimethyl ammonium hydroxide (CTAOH) BGE, titrated with benzenetricarboxylic acid (BTC) to pH=8.54, run on a 50 :m I.D. bare fused silica capillary, Lₜ=25.7 cm, L_{d}=19.3 cm at 25 kV, negative to positive polarity, T=25 EC, UV detector set at 214 nm indicated that the residual lithium and sulfate ion concentrations were lower than the detection limits.

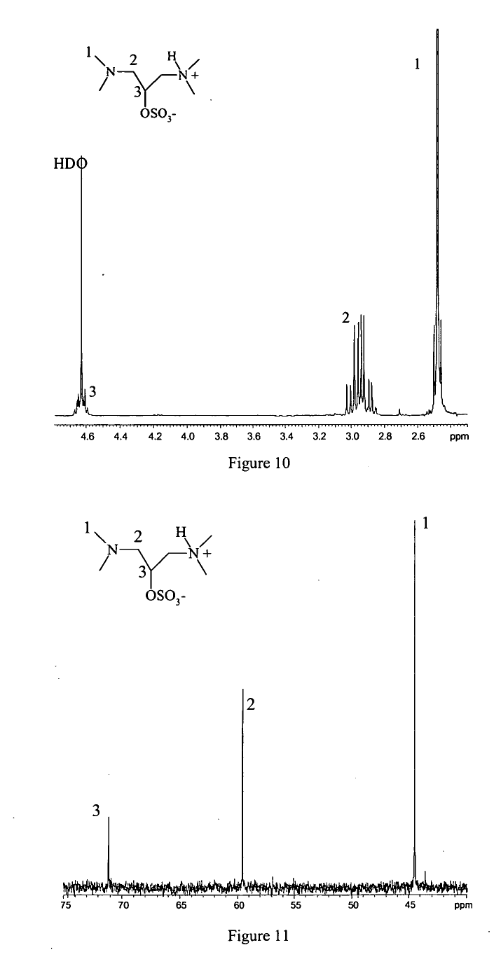

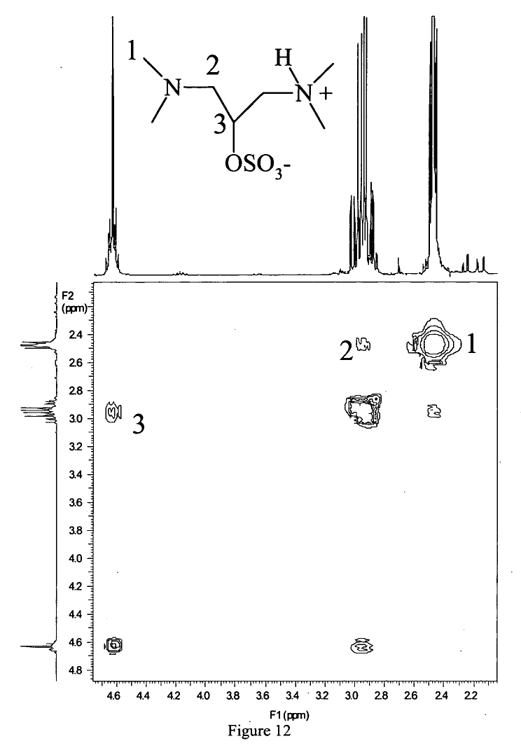

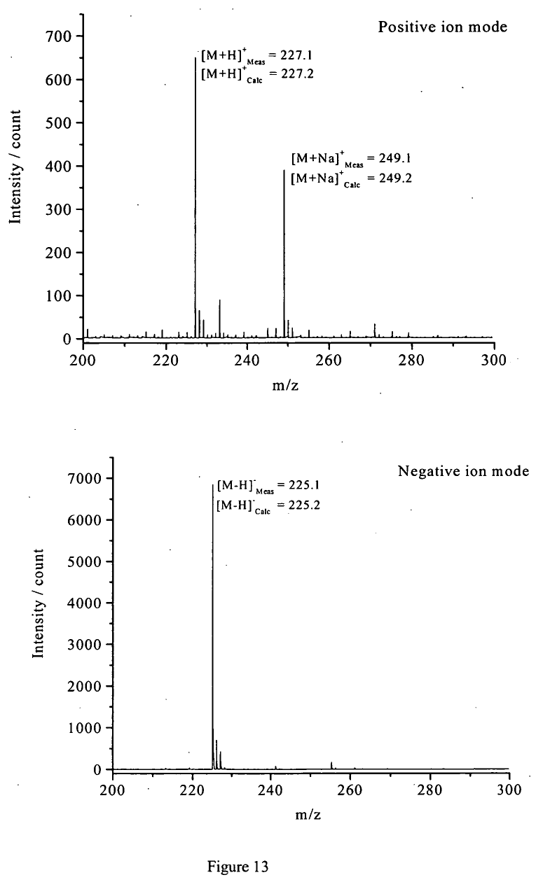

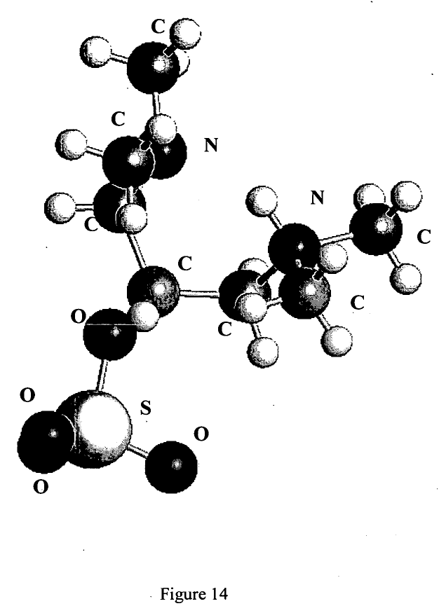

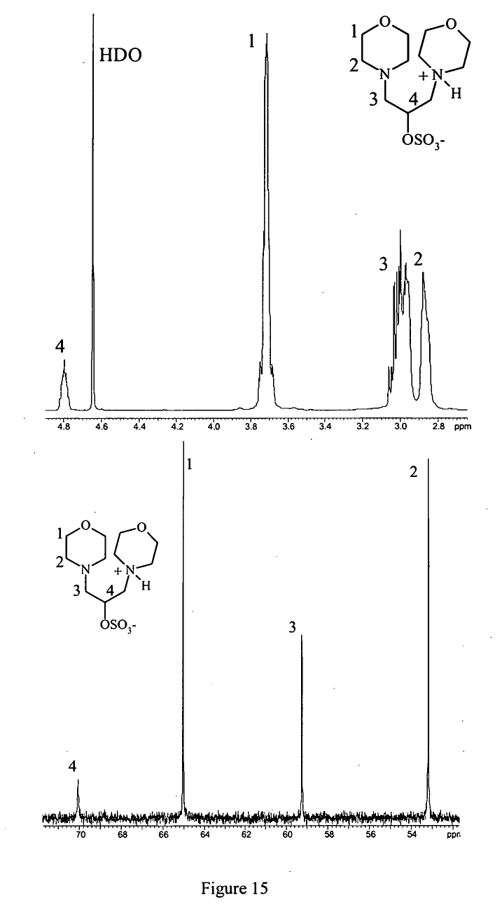

The final solid product was characterized by ¹H- and ¹³C-NMR, ¹H-¹H COSY and ¹H-¹³C HETCOR NMR spectroscopy (FIGS. 10-12). The identity and purity of the final product were further confirmed by high resolution ESI-MS. The ESI-MS spectrum in the positive ion mode is shown in FIG. 13. Only signals corresponding to BDASP and its Na⁺ adduct are seen. Single crystals of the product were grown by slow and undisturbed cooling of a concentrated aqueous solution of BDASP and its X-ray crystal structure was determined. An image of the X-ray crystal structure of BDASP is shown in FIG. 14.

### EXAMPLE 3

Synthesis of 1,3-dimorpholino-2-O-sulfo-propane (DMSP): fit a clean, 3 L, three-neck round bottom flask with an ice-water cooled condenser. Add 750 g (8.61 mol) morpholine (MOR) to the flask, set the flask in an oil bath. Mix together 265.5 g (2.87 mol) epichlorohydrin (EH) and 500 mL tetrahydrofuran (THF). Slowly add the EH solution into the flask and heat to 65° C., while stirring the solution with a mechanical stirrer. Take samples periodically and monitor the increase in the amount of 1,3-dimorpholino-2-propanol (DMP) and the corresponding decrease in the amount of the amino alcohol intermediate by NMR. Depending on whether more MOR or more EH is needed, add the corresponding amount into the reaction flask. Stirring continuously, heat the reaction mixture for 22 hours. At the end of the reaction, take the flask off of the oil bath and set it to cool on a cork O-ring with paper towels underneath (to absorb the oil).

Into the cool reaction mixture, add enough acetone to form a dilute slurry. Filter the slurry using a Buchner funnel and a suction flask. Remove acetone and THF under reduced pressure.

Fit a clean, 5 L, three-neck round bottom flask with an ice-water cooled condenser. Add 500 mL DMF to the flask, set the flask in an oil bath. Add the processed DMP from step 1 into the flask and heat it to 65° C. while stirring the solution with a mechanical stirrer. To the warm, stirring solution add 466.1 g (2.87 mol) SO₃XPyr and continuously heat and stir the mixture at 65° C. for 4 hours. Take the flask off of the oil bath and set it to cool on a cork O-ring with paper towels underneath (to absorb the oil).

To the cooled reaction mixture, add 700 mL DMF and with the aid of a mechanical stirring rod and a spatula, manually dislodge the solids and remove the chunks from the round bottom flask into a 5 L beaker. Add 1 L DMF into the beaker and make a slurry of the solids. Filter the slurry using a Buchner funnel and a suction flask. Remove the solid cake and filter paper from the funnel and add them into another 5 L beaker and make a slurry with another 1 L DMF. Repeat the process until the DMF filtrate comes out pale yellow. Then, remove the solid cake and filter paper from the funnel and add them into another 5 L beaker and make a slurry with 1 L of acetone. Filter the slurry, and make another slurry of the solids in 1 L of ethanol, and filter again. Let the solids dry inside the fume hood.

Split the solids into two batches and dissolve each batch in a minimum volume (around 2 L) of deionized water. Measure the pH of the solution and titrate it to pH=5.8 using a 10% LiOH solution (if the solution pH is below 5.8) or 2M H₂SO₄ (if the solution pH is above 5.8). Remove about half of the water using a rotovap with the water bath set at 65° C. Let the solution cool in the fume hood with continuous stirring for 24 hours. Filter the slurry, wash the solid cake with cold ethanol three times and let the cake dry in the fume hood. Analyze both the crystallization mother liquor and the solids by CE and repeat crystallization from deionized water if necessary.

Combine the two crystallized batches and recrystallize the combined solids from deionized water. Filter off the first crop of crystals, wash the cake with cold ethanol three times, let the solids dry and analyze them by CE. Remove more water from the crystallization mother liquor to get a second crop of crystals. Analyze each crop of crystals, and combine the similar ones. Repeat the concentration followed by crystallization steps until co-crystallization of DMSP and any of the salt contaminants is observed.

The pI of DMSP was determined by indirect UV detection CE. Using a 26 :m I.D. bare fused silica capillary, Lₜ=26.1 cm, L_{d}=19.7 cm at 15 kV in positive to negative polarity, T=25 EC, and the UV detector set at 214 nm, conventional CE was carried out in 20 mM acetic acid BGEs titrated with imidazole and 20 mM pyridine BGEs titrated with pTSA. The pI of DMSP was determined to be 5.8.

Conventional indirect UV-detection CE using a 20 mM acetic acid BGE titrated with imidazole to pH=4.5, run on a 26 :m I.D. bare fused silica capillary, Lₜ=26.5 cm, L_{d}=19.7 cm at 25 kV, positive to negative polarity, T=25 EC, UV detector set at 214 nm, and a 20 mM Tris, 0.1 mM CTAOH BGE titrated to pH=8.5 with BTC, run on a 50 :m I.D. bare fused silica capillary, Lₜ=25.7 cm, L_{d}=19.3 cm at 10 kV, negative to positive polarity, T=25 EC, UV detector set at 214 nm indicated that the residual lithium and sulfate ion concentrations were below the detection limits.

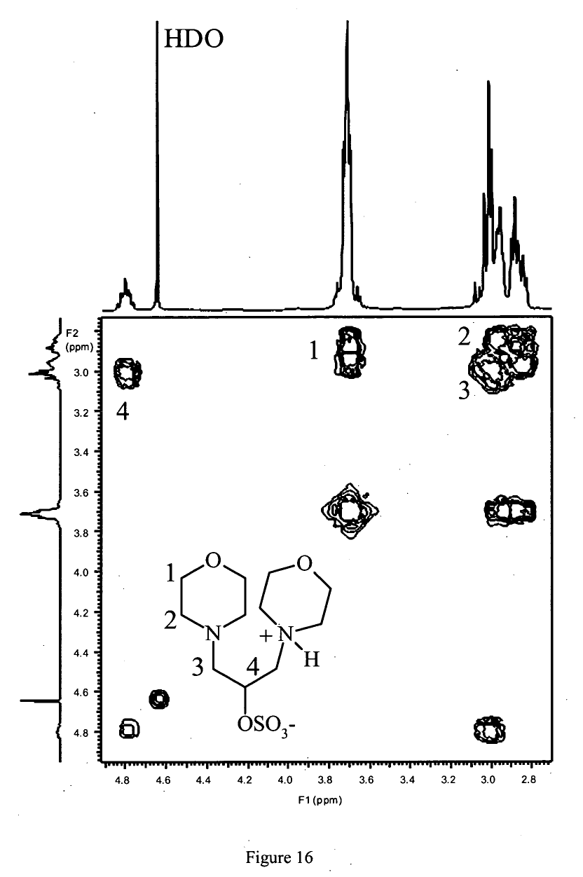

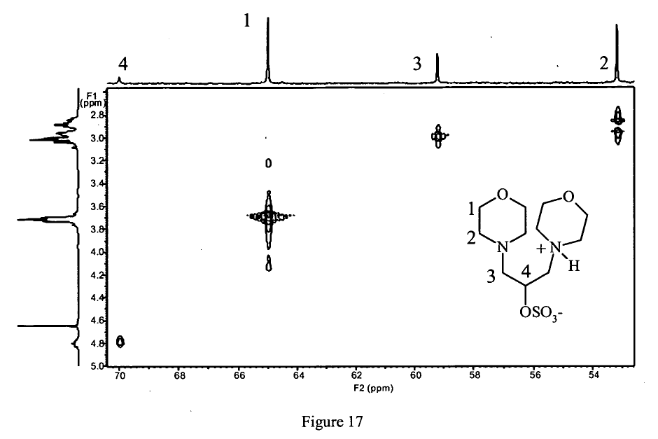

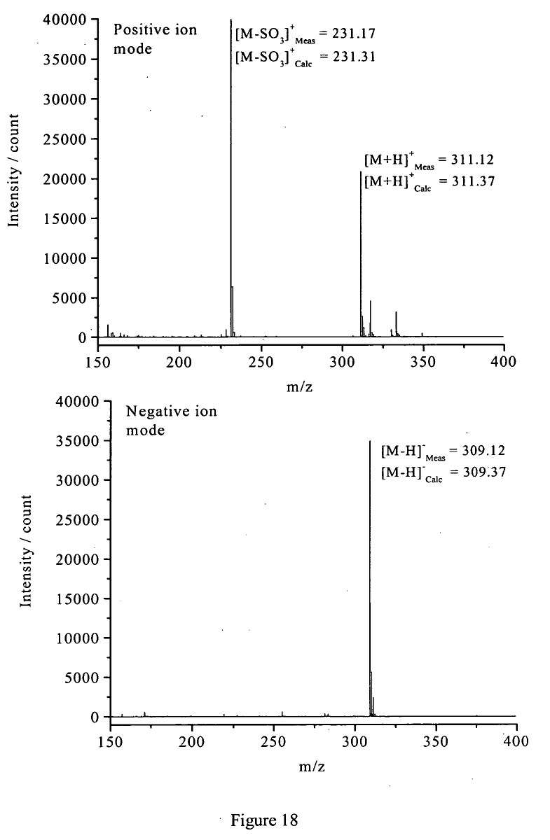

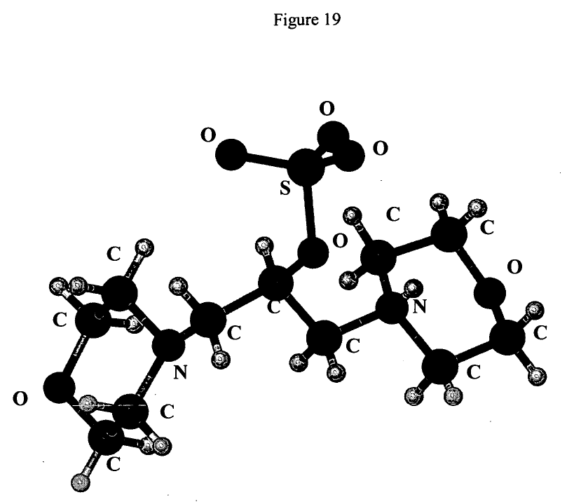

FIGS. 15, 16 and 17 show the final product as characterized by ¹H- and ¹³C-NMR, ¹H-¹H COSY and ¹H-¹³C HETCOR NMR spectroscopy, respectively. The identity and purity of the final product were further confirmed by high resolution ESI-MS. FIG. 18 shows the ESI-MS spectra in the positive and negative ion modes. Only signals corresponding to DMSP (and the corresponding adducts and fragment ions) are seen. Single crystals of DMSP were grown by slow and undisturbed cooling of a concentrated aqueous solution of DMSP and its X-ray crystal structure was determined. A ball-and-stick image of the X-ray crystal structure is shown in FIG. 19.

### EXAMPLE 4

Synthesis of 1,3-bis(dipropylamino)-2-O-sulfo-propane (BDPSP): 7.5 g (0.074 mol) dipropylamine (DPA) was added into a 50 mL round bottom flask fitted with an ice-water cooled condenser. 2.3 g (0.025 mol) EH was mixed with 5 mL THF and the solution was added into the flask with DPA. The flask was warmed in an oil bath set at 65° C. Heavy solid formation was observed within 1 hour of heating. After 22 hours of heating, the reaction mixture was cooled. Acetone was added to the 1,3-bis(dipropylamino)-2-propanol (BDPP) reaction mixture and the slurry was filtered. Acetone was removed from the filtrate under reduced pressure to yield a yellow, viscous liquid.

A clean, 50 mL, three-neck round bottom flask was fitted with an ice-water cooled condenser. 10 mL DMF was added to the flask. 5 g (0.022 mol) BDPP was added and the flask was warmed in an oil bath to 65° C. 3.9 g (0.025 mol) SO₃XPyr was added to the warm solution and the mixture was stirred and heated continuously at 650° C. for 4 hours. Heavy solid formation was observed after 1 hour of heating. After 4 hours of heating, the mixture was allowed to cool. 5 mL DMF was added to the solids to make a slurry. The slurry was filtered and the solids were suspended in 50 mL acetone, and the acetone slurry was filtered. The solids were then dispersed in 50 mL ethyl alcohol followed by filtration of the slurry to obtain an off-white colored cake.

The pI of BDPSP was determined by indirect UV detection CE using a 26 :m I.D. bare fused silica capillary, Lₜ=26.1 cm, L_{d}=19.7 cm at 15 kV in positive to negative polarity, T=25 EC, UV detector set at 214 nm, 20 mM Tris and 20 mM benzylamine BGEs titrated with pTSA. The pI of BDPSP was determined to be 8.7.

Conventional indirect UV-detection CE using a 20 mM acetic acid BGE titrated with imidazole to pH=4.5, run on a 26 :m I.D. bare fused silica capillary, Lₜ=26.5 cm, L_{d}=19.7 cm at 25 kV, positive to negative polarity, T=25 EC, UV detector set at 214 nm, and a 20 mM Tris, 0.1 mM CTAOH BGE titrated with BTC to pH=8.5 BGE, run on a 50 :m I.D. bare fused silica capillary, Lₜ=25.7 cm, L_{d}=19.3 cm at 10 kV, negative to positive polarity, T=25 EC, UV detector set at 214 nm indicated that residual lithium and sulfate ion concentrations were below the detection limits.

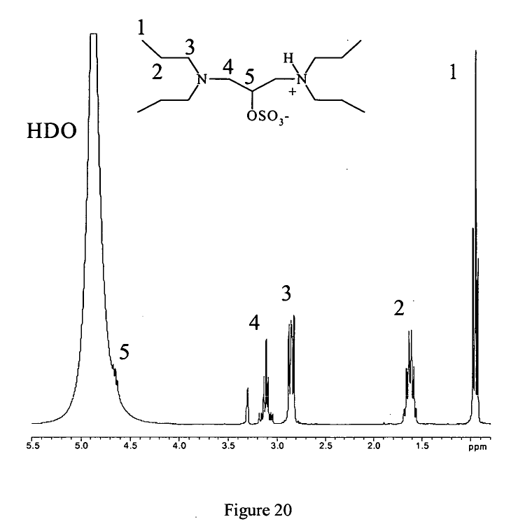

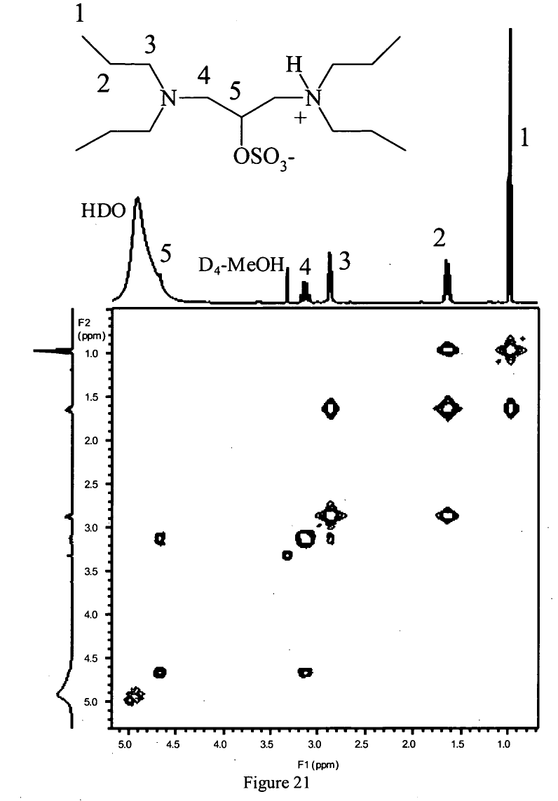

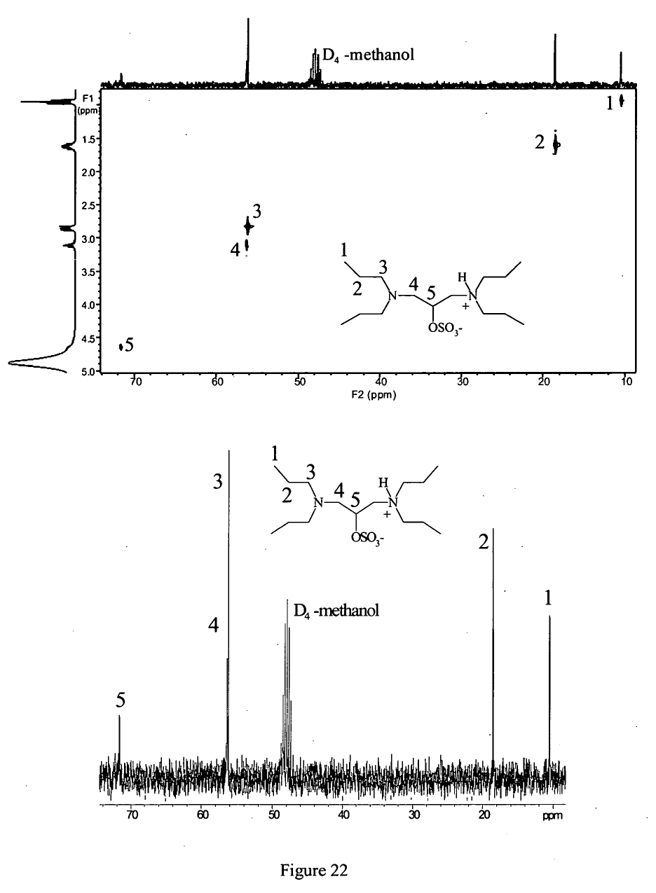

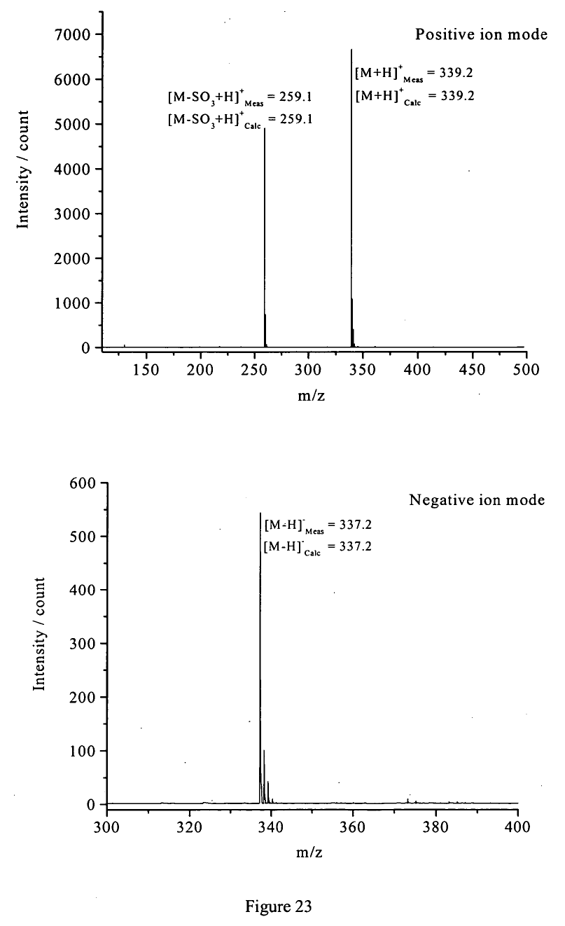

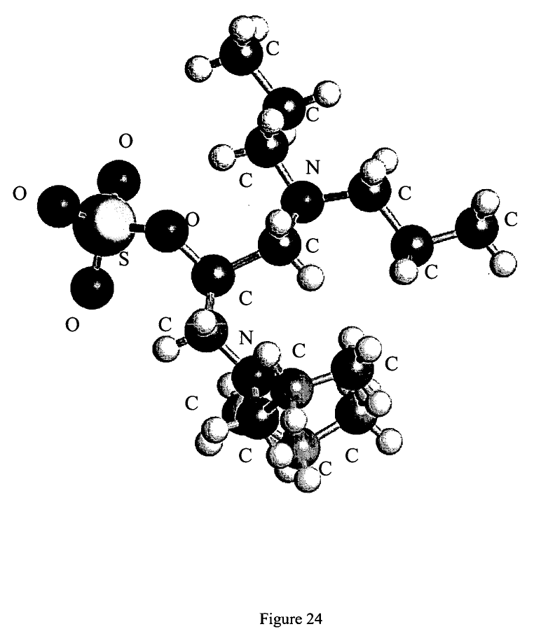

The final product was characterized by ¹H- and ¹³C-NMR, ¹H-¹H COSY and ¹H-¹³C HETCOR NMR spectroscopy (FIGS. 20-22). The identity and purity of the final product were further confirmed by high resolution ESI-MS. The ESI-MS spectra in the positive and negative ion modes are shown in FIG. 23. Only signals corresponding to BDPSP (and the corresponding fragment ions) are seen. Single crystals of BDPSP were grown by slow and undisturbed cooling of a concentrated aqueous solution of BDPSP and its X-ray crystal structure was determined. A ball-and-stick image of the X-ray crystal structure is shown in FIG. 24.

### EXAMPLE 5

Synthesis of 1,3-dipiperidino-2-O-sulfo-propane (DPSP): Fit a clean, 1 L, three-neck round bottom flask with an ice-water cooled condenser. Add 90 g (1.06 mol) piperidine (PIP) to the flask, set the flask in an oil bath over a magnetic stirrer. Mix together 32.6 g (0.352 mol) EH and 100 mL THF. Slowly, add the EH solution into the flask and heat to 65° C. while stirring the solution with a one-inch football-shaped magnetic stir bar. Take samples periodically and monitor the increase in the amount of DPP (and the corresponding decrease in the amount of the amino alcohol intermediate). Depending on whether more PIP or more EH is needed, add the corresponding amount into the reaction flask. Stirring continuously, heat the reaction mixture for 22 hours. At the end of the reaction, take the flask off of the oil bath and set it to cool on a cork O-ring with paper towels underneath (to absorb the oil).

Into the cool reaction mixture, add enough acetone to form a dilute slurry. Filter the slurry using a Buchner funnel and a suction flask. Remove acetone and THF under reduced pressure.

Fit a clean, 1 L, three-neck round bottom flask with an ice-water cooled condenser. Add 200 mL DMF to the flask, set the flask in an oil bath. Add DPP into the flask and heat to 65° C. while stirring the solution with a mechanical stirrer. To the warm, stirring solution add 61.6 g (0.387 mol) SO₃XPyr and continuously heat and stir the mixture at 65° C. for 4 hours. Take the flask off of the oil bath and set it to cool on a cork O-ring with paper towels underneath (to absorb the oil).

To the cooled reaction mixture, add 200 mL DMF and with the aid of a spatula, manually dislodge the solids and remove the chunks from the round bottom flask into a 2 L beaker. Add 300 mL DMF into the beaker and make a slurry of the solids. Filter the slurry using a Buchner funnel and a suction flask. Remove the solid cake and filter paper from the funnel and add them into another 2 L beaker and make a slurry with another 200 mL DMF. Repeat the steps until the DMF filtrate becomes pale yellow. Then, remove the solid cake and filter paper from the funnel and add them into another 2 L beaker and make a slurry with 300 mL of acetone. Filter the slurry, and make another slurry of the solids in 200 mL of ethanol, and filter again. Let the solids dry inside the fume hood.

Dissolve the solids in a minimum volume (around 250 mL) of deionized water. Measure the pH of the solution and titrate it to pH=8.9 using a 10% LiOH solution (if the solution pH is below 8.9) or 2M H₂SO₄ (if the solution pH is above 8.9). Remove about half of the water using a rotovap with the water bath set at 65° C. Let the solution cool in the fume hood with continuous stirring for 24 hours. Filter the slurry, wash the solid cake with ice-cold ethanol three times and let the cake dry in the fume hood. Analyze both the crystallization mother liquor and the solids by CE and repeat the crystallization from deionized water if necessary.

Remove more water from the crystallization mother liquor to get a second crop of crystals. Analyze each crop of crystals, and combine the similar ones. Repeat the concentration followed by crystallization steps until co-crystallization of DPSP and any of the salt contaminants is observed.

The pI of DPSP was determined by indirect UV detection CE using a 26 :m I.D. bare fused silica capillary, Lₜ=26.1 cm, L_{d}=19.7 cm at 15 kV in positive to negative polarity, T=25 EC, UV detector set at 214 nm, 20 mM Tris and 20 mM benzylamine BGEs titrated with pTSA. The pI of DPSP was determined to be 8.9.

Conventional indirect UV detection CE with a 20 mM acetic acid BGE titrated with imidazole to pH=4.5, run on a 26 :m I.D. bare fused silica capillary, Lₜ=26.5 cm, L_{d}=19.7 cm at 25 kV, positive to negative polarity, T=25 EC, UV detector set at 214 nm, and a 20 mM Tris, 0.1 mM CTAOH BGE titrated to pH=8.5 with BTC, run on a 50 :m I.D. bare fused silica capillary, Lₜ=25.7 cm, L_{d}=19.3 cm at 10 kV, negative to positive polarity, T=25 EC, UV detector set at 214 nm indicated that the residual lithium and sulfate ion concentrations were below the detection levels.

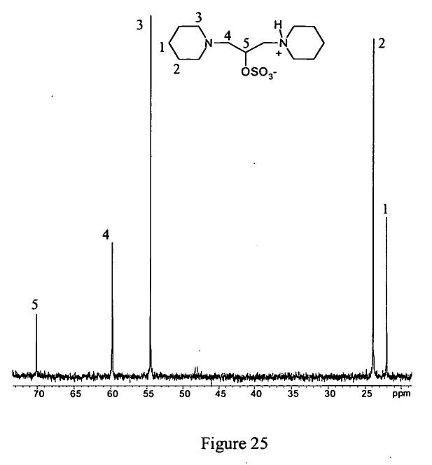

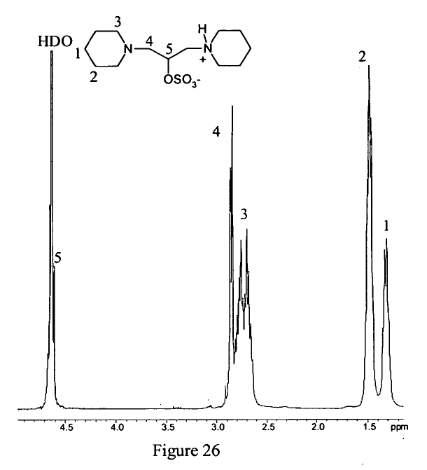

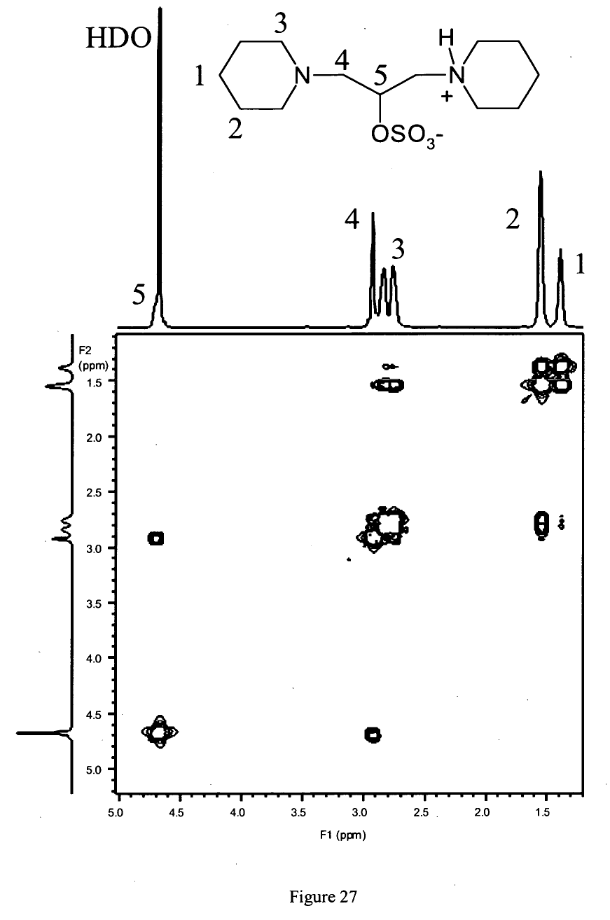

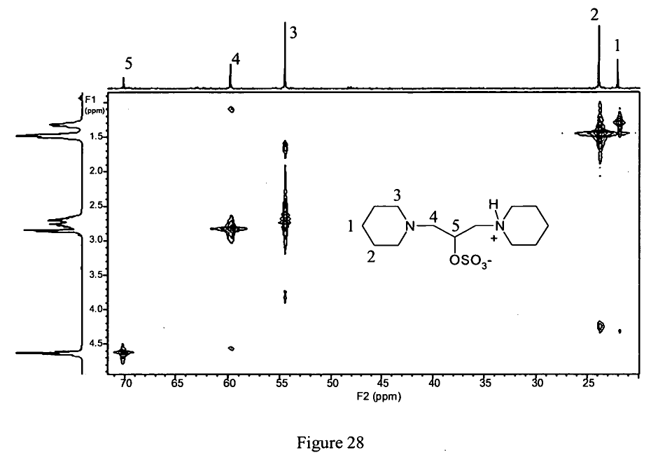

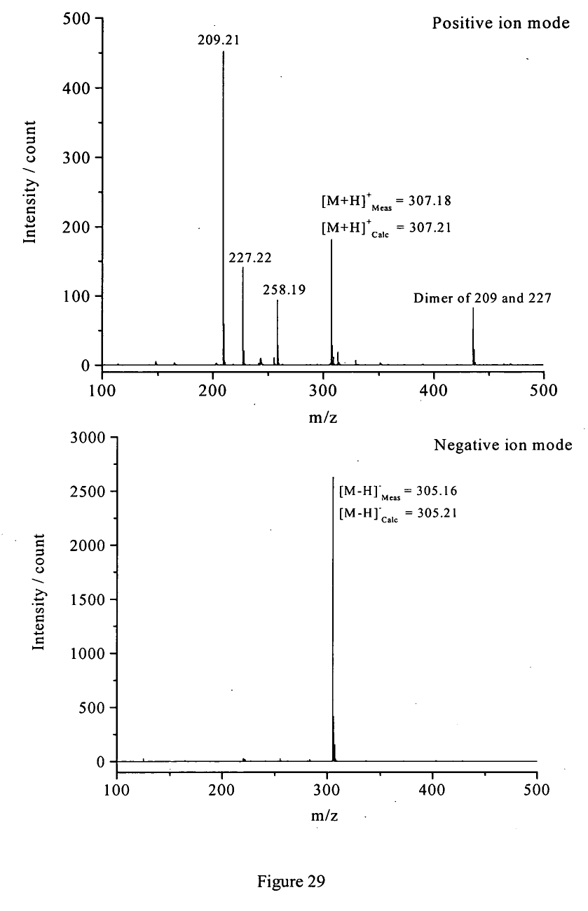

The final product was characterized by ¹H- and ¹³C-NMR, ¹H-¹H COSY and ¹H-¹³C HETCOR NMR spectroscopy (FIGS. 25-27). The identity and purity of the final product were further confirmed by high resolution ESI-MS. The ESI-MS spectra in the positive and negative ion modes are shown in FIG. 28. Only signals corresponding to DPSP and its fragment ions are seen.

The conductivities, pH values and buffering capacities of 50 mM solutions of some of the ampholytic buffers of the present invention are listed in Table 1. For comparison purposes, values for histidine (HIS), a widely used low conductivity ampholytic buffer are also included.

TABLE 1

| | | | | Buffering |
|---|---|---|---|---|
| Ampholytic | Concentration | Conductivity | | capacity |
| buffer | (mM) | (mS/m) | pH | (mM/pH) |
| BCPDEAH | 50 | 18.4 | 4.15 | 35.7 |
| DMSP | 50 | 7.3 | 5.79 | 14.7 |
| BDAPS | 50 | 25.8 | 7.82 | 25.0 |
| DPSP | 50 | 18.1 | 8.35 | 20.8 |
| HIS | 50 | 3.5 | 7.44 | 12.5 |

In the claims, all transitional phrases such as “comprising,” “including,” “carrying,” “having,” “containing,” “involving,” and the like are to be understood to be open-ended, i.e., to mean including but not limited to. Only the transitional phrases “consisting of” and “consisting essentially of,” respectively, shall be closed or semi-closed transitional phrases.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

[1] Ivory, C. F., Sep. Sci. Technology 2000, 35, 1777-1793.
[2] Krivankova, L., Bocek, P., Electrophoresis 1998, 19, 1055-1062.
[3] Simpson, R. J., Proteins and Proteomics: A Laboratory Manual, Cold Spring Harbor Laboratory Press, Cold Spring Harbor, N.Y., 2003.
[4] Vesterberg, O., Svensson, H., Acta. Chem. Scand. 1966, 20, 820-834.
[5] Williams, R. R., Waterman, R. E., Proc. Soc. Exptl. Biol. Med. 1929, 27, 56-59.
[6] Kolin, A., J. Chem. Phys. 1955, 23, 407-410.
[7] Martin, A. J. P., Hampson, F., U.S. Pat. No. 4,243,507, 1981.
[8] Faupel, M., Barzaghi, B., Gelfi, C., Righetti, P. G., J. Biochem. Biophys. Methods 1987, 15, 147-161.
[9] IsoPrime Manual, Amersham Pharmacia Biotech., San Francisco, Calif., 1999.
[10] Bjellqvist, B., Ek, K., Righetti, P. G., Gianazza, E., Gorg, A., Westermeier, R., Postel, W., J. Biochem. Biophys. Methods 1982, 6, 317-339.
[11] Dossi, G., Celentano, F., Gianazza, E., Righetti, P. G., J. Biochem. Biophys. Methods 1983, 7, 123-142.
[12] Righetti, P. G., Laboratory Techniques in Biochemistry and Molecular Biology, Vol. 20, Elsevier Science Publishing Company, Inc., New York, N.Y., 1990.
[13] Righetti, P. G., Barzaghi, B., Faupel, M., J. Biochem. Biophys. Methods 1987, 15, 163-176.
[14] Barzaghi, B., Righetti, P. G., Faupel, M., J. Biochem. Biophys. Methods 1987, 15, 177-188.
[15] Righetti, P. G., Barzaghi, B., Luzzana, M., Manfredi, G., Faupel, M., J. Biochem. Biophys. Methods 1987, 15, 189-198.
[16] Glukhovskiy, P, Landers, T. A., Vigh, Gy., Electrophoresis 2000, 21, 762-766.
[17] Shave, E., Vigh, Gy., J. Chromatogr. A, 2002, 989, 73-78.
[18] Herbert, B., Righetti, P. G., Electrophoresis 2001, 21, 3639-3648.
[19] Zou, X., Speicher, D. W., Anal. Biochem. 2000, 284, 266-278.
[20] Invitrogen Corp., Dec. 18, 2003, http://www.invitrogen.com/content.cfm?pageid=9719&CMP═ILC-ZOOMFRACTIONATOR&GPN=LAST.
[21] Proteome Systems Ltd., Dec. 15, 2003, http://www.proteomesystems.com/Catalogue/Profile.asp? ProductID=74
[22] Ogle, D., Ho, A., Gibson, T., Rylatt, D. B., Shave, E., Lim, P., Vigh, Gy., J. Chromatogr. A 2002, 979, 155-161.
[23] Cretich, M., Pirri, G., Carrea, G., Chiari, M., Electrophoresis 2003, 24, 577-581.
[24] McMurry, J., Organic Chemistry, Brooks/Cole, Pacific Grove, Calif., 2000.
[25] Chiari, M., Nesi, M., Roncada, P., Righetti, P. G., Electrophoresis 1994, 15, 953-959.
[26] Shave, E., Vigh, Gy., Electrophoresis 2004, 25, 381-387.
[27] Horvath, Zs., Corthals, G. L., Wrigley, C. W., Margolis, J., Electrophoresis 1994, 15, 968-971.
[28] Fullarton, J. R., Kenny, A. J., Biochem. J. 1970, 116, 147-149.
[29] Hjertén, S., Liao, J., U.S. Pat. No. 5,464,517, 1995.
[30] Stoyanov, A., Righetti, P. G., J. Chromatogr. A 1997, 169-176.
[31] Righetti, P. G., Bossi, A., Gelfi, C., J. Cap. Elec. 1997, 4, 47-59.
[32] Stoyanov, A., Righetti, P. G., Electrophoresis 1998, 19, 1674-1676.
[33] Bossi, A., Olivieri, E., Castelletti, L., Gelfi, C., Hamdan, M., Righetti, P. G., J. Chromatogr. A 1999, 853, 71-82.

What is claimed is:

1. An ampholytic buffer molecule comprising a first weakly acidic functional group, a second weakly acidic functional group, and a weakly basic or a cationic functional group wherein the first weakly acidic functional group has a first pKa value and a first charge-carrying or chargable atom, the second weakly acidic functional group has a second pKa value and a second charge-carrying or chargable atom, the weakly basic functional group or the cationic functional group has a third charge-carrying or chargable atom, the first weakly acidic functional group and the second weakly acidic functional group are similar or different in structure, the first and the second charge-carrying or chargable atoms are similar or different, the molecule has a unique pI value, the pI value lies between the first pKa value and the second pKa value, an absolute value of a difference between the first and the second pKa values is less than 3, the first and the second and the third charge-carrying or chargable atoms are four or more covalent bonds away from each other, and wherein the molecule in a solution has an isoelectric state with a high buffering capacity and a high conductivity.

2. The molecule of claim 1, wherein the pKa values are between 1 and 14.

3. The molecule of claim 1, wherein the cationic functional group is a quaternary ammonium group.

4. An ampholytic buffer molecule comprising a first weakly basic functional group, a second weakly basic functional group, and a weakly acidic or an anionic functional group wherein the first weakly basic functional group has a first charge-carrying or chargable atom, the second weakly basic functional group has a second charge-carrying or chargable atom, the weakly acidic functional group or the anionic functional group has a third charge-carrying or chargable atom, the first weakly basic functional group has a first conjugate acid form with a first pKa value, the second weakly basic functional group has a second conjugate acid form with a second pKa value, the first weakly basic functional group and the second weakly basic functional group are similar or different in structure, the first and the second charge-carrying or chargable atoms are similar or different, the molecule has a unique pI value, the pI value lies between the first pKa value and the second pKa value, an absolute value of a difference between the first and the second pKa values is less than 3, the first and the second and the third charge-carrying or chargable atoms are four or more covalent bonds away from each other, wherein the molecule in a solution has an isoelectric state with a high buffering capacity and a high conductivity.

5. The molecule of claim 4, wherein the pKa values are between 1 and 14.

6. The molecule of claim 4, wherein the anionic functional group is a sulfate or sulfonate group.

7. An ampholytic buffer molecule comprising a weakly acidic functional group and a weakly basic functional group, wherein the weakly acidic functional group has a first charge-carrying or chargable atom, the weakly basic functional group has a second charge-carrying or chargable atom, the weakly acidic functional group has a first pKa value, the weakly basic functional group has a conjugate acid form with a second pKa value, wherein the pI value is between the first pKa value and the second pKa value, an absolute value of a difference between the first and the second pKa values is less than 3, the first and the second charge-carrying or chargable atoms are four or more covalent bonds away from each other, wherein the molecule in a solution has an isoelectric state with a high buffering capacity and a high conductivity.

8. The molecule of claim 7, wherein the pKa values are between 1 and 14.

9. A method of making an ampholytic buffer molecule comprising the steps of:
reacting a secondary amine with a first ester formed from a first weak acid, the first weak acid having a pKa value between 1 and 14, the first weak acid having a functional group capable of reacting with the secondary amine, the reaction resulting in an ester-group bearing tertiary amine,
reacting the ester-group bearing tertiary amine with a second ester formed from a 25 second weak acid, the second weak acid having a pKa value between 1 and 14, the second weak acid having a functional group capable of reacting with the ester-group bearing tertiary amine, the reaction resulting in a quaternary ammonium compound bearing two ester groups, the first weak acid and the second weak acid having a similar or a different structure, the first ester and the second ester having a similar or a different structure,
hydrolyzing the first ester group and the second ester group of the quaternary ammonium compound bearing two ester groups forming an ampholytic compound bearing a first weakly acidic functional group having a charge-carrying or chargable atom and a second weakly acidic functional group having a charge-carrying or chargable atom, four or more bonds separating the charged or chargeable atoms of the first weakly acidic functional group and the second weakly acidic functional group and a nitrogen atom of the quaternary ammonium group, and
recovering, in a pure isoelectric form, the ampholytic buffer molecule.

10. A method of making an ampholytic buffer molecule comprising the steps of:
reacting a primary amine with a first ester formed from a first weak acid, the first weak acid having a pKa value between 1 and 14, the first weak acid having a functional group capable of reacting with the primary amine, the reaction resulting in an ester-group bearing secondary amine,
reacting the ester-group bearing secondary amine with a second ester formed from a second weak acid, the second weak acid having a pKa value between 1 and 14, the second weak acid having a functional group capable of reacting with the ester-group bearing secondary amine, the reaction resulting in a tertiary amine bearing two ester groups, the first weak acid and the second weak acid having a similar or a different structure, the first ester and the second ester having a similar or different structure,
hydrolyzing the first ester group and the second ester group of the tertiary amine compound bearing two ester groups forming an ampholytic compound bearing a first weakly acidic functional group having a charge-carrying or chargable atom and a second weakly acidic functional group having a charge-carrying or chargable atom, four or more bonds separating the charged or chargeable atoms of the first weakly acidic functional group and the second weakly acidic functional group and a nitrogen atom of the tertiary ammonium group, and
recovering, in a pure isoelectric form, the ampholytic buffer molecule.

11. A method of making an ampholytic buffer molecule comprising the steps of:
reacting a primary amine with a first ester formed from a first weak acid, the first weak acid having a pKa value between 1 and 14, the first weak acid having a functional group capable of reacting with the primary amine, the reaction resulting in an ester-group bearing secondary amine,
reacting the ester-group bearing secondary amine with a second ester formed from a second weak acid, the second weak acid having a pKa value between 1 and 14, the second weak acid having a functional group capable of reacting with the ester-group bearing secondary amine, the reaction resulting in a tertiary amine bearing two ester groups, the first weak acid and the second weak acid having a similar or a different structure, the first ester and the second ester heaving a similar or different structure,
reacting the tertiary amine bearing two ester groups with a reagent having a functional group capable of reacting with the tertiary amine bearing two ester groups, the reaction resulting in a quaternary ammonium compound bearing two ester groups,
hydrolyzing the first ester group and the second ester group of the quaternary ammonium compound bearing two ester groups forming an ampholytic compound bearing a first weakly acidic functional group having a charge-carrying or chargable atom and a second weakly acidic functional group having a charge-carrying or chargable atom, four or more bonds separating the charged or chargeable atoms of the first weakly acidic functional group and the second weakly acidic functional group and a nitrogen atom of the quaternary ammonium group, and
recovering, in a pure isoelectric form, the ampholytic buffer molecule.

12. A method of making an ampholytic buffer molecule comprising the steps of:
reacting a primary amine with a first ester formed from a first weak acid, the first weak acid having a pKa value between 1 and 14, the first weak acid having a functional group capable of reacting with the primary amine, the reaction resulting in an ester-group bearing secondary amine;
reacting the ester-group bearing secondary amine with a reagent having a functional group capable of reacting with the ester-group bearing secondary amine, the reaction resulting in a tertiary amine bearing an ester group;
reacting the tertiary amine bearing an ester group with a second ester formed from a second weak acid, the second weak acid having a pKa value between 1 and 14, the second weak acid having a functional group capable of reacting with the tertiary amine bearing an ester group, the reaction resulting in a quaternary ammonium compound bearing two ester groups, the first weak acid and the second weak acid having a similar or a different structure, the first ester and the second ester having a similar or different structure;
hydrolyzing the first ester group and the second ester group of the quaternary ammonium compound bearing two ester groups forming an ampholytic compound bearing a first weakly acidic functional group having a charge-carrying or chargable atom and a second weakly acidic functional group having a charge-carrying or chargable atom, four or more bonds separating the charged or chargeable atoms of the first weakly acidic functional group and the second weakly acidic functional group and a nitrogen atom of the quaternary ammonium group, and
recovering, in a pure isoelectric form, the ampholytic buffer molecule.

13. A method of making an ampholytic buffer molecule comprising the steps of:
selecting a diamino alcohol having a first weakly basic functional group and a second weakly basic functional group and a hydroxyl group, the first weakly basic functional group having a conjugate acid form with a pKa value between 1 and 14, the second weakly basic functional group having a conjugate acid form with a pKa value between 1 and 14, the first and the second weakly basic functional groups having a similar or a different structure;
converting the hydroxyl group of the diamino alcohol into a weakly acidic functional group or an anionic functional group having a charge-carrying or chargable atom forming an ampholytic compound bearing a first weakly basic functional group having a charge-carrying or chargable atom and a second weakly basic functional group having a charge-carrying or chargable atom, four or more bonds separating the charged or chargeable atoms of the first weakly basic functional group and the second weakly basic functional group and the weakly acidic or anionic functional group, and
recovering, in a pure isoelectric form, the ampholytic buffer molecule.

14. A method of making an ampholytic buffer molecule comprising the steps of:
selecting a first secondary amine having a conjugate acid form with a pKa value between 1 and 14;
selecting a second secondary amine having a conjugate acid form with a pKa value between 1 and 14, the first and the second secondary amines having a similar or different structure;
selecting a difunctional reagent having a first reactive group, a second reactive group and a hydroxyl group or a protected hydroxyl group, the first and the second reactive groups having a similar or different structure;
reacting, simultaneously or sequentially, the first secondary amine with the first reactive group of the difunctional reagent and the second secondary amine with the second reactive group of the difunctional reagent forming a diamino compound and preserving, intact, the hydroxyl group or protected hydroxyl group;
converting the hydroxyl group or the protected hydroxyl group of the diamino compound into a weakly acidic functional group or an anionic functional group having a charge-carryig or chargable atom forming an ampholytic compound having four or more bonds between the charge-carrying or chargeable atoms of the first weakly basic functional group and the second weakly basic functional group and the weakly acidic or anionic functional group; and
recovering, in a pure isoelectric form, the ampholytic buffer molecule.

15. A compound of the formula: 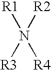
R1 and R2 are each, independently, selected from a group comprising an unsubstituted or substituted alkyl group, aryl group, alkylaryl group, oxyalkyl group, oxyaryl group and oxyalkylaryl group;
R3 and R4 are each, independently, selected from a group comprising an unsubstituted or substituted carboxyalkyl group, carboxyaryl group, carboxyalkylaryl group, carboxyalkyloxy group, carboxyaryloxy group, and caboxyalkylaryloxy group; and
the compound has a high buffering capacity and a high conductivity in isoelectric state.

16. The compound of claim 15, wherein the R1 and R2 groups are substituted alkyl groups of 1 to 6 carbon atoms substituted with one or more alkyl groups, one or more alkoxy groups, one or more hydroxy groups, with one or more hydroxyalkyl groups, one or more ester groups, one or more oxo groups, one or more nitro groups, one or more CN groups, or substituted aryl groups of 6 to 14 carbon atoms substituted with one or more alkyl groups, one or more alkoxy groups, one or more hydroxyalkyl groups, one or more ester groups, one or more oxo groups, one or more nitro groups, one or more CN groups, or substituted alkylaryl groups of 7 to 24 carbon atoms substituted with one or more alkyl groups, one or more alkoxy groups, one or more hydroxyalkyl groups, one or more ester groups, one or more oxo groups, one or more nitro groups, one or more CN groups.

17. The compound of claim 15, wherein R3 and R4 are each, independently, selected from one of the group comprising of carboxypropyl groups, carboxybutyl groups, carboxyamyl groups and carboxyhexyl groups or from the group comprising of carboxyphenyl groups, dicarboxyphenyl groups, tricarboxyphenyl groups, tetracarboxyphenyl groups and pentacarboxyphenyl groups or from the group comprising of carboxymethylphenyl groups, carboxyethylphenyl groups and carboxypropylphenyl groups or from the group comprising of methylcarboxyphenyl groups, ethylcarboxyphenyl groups and propylcarboxyphenyl groups or combinations thereof.

18. The compound of claim 15, wherein R1 and R2 are each, independently ethyl groups; and R3 and R4 are each, independently carboxypropyl groups.

19. The compound of claim 15, wherein R1 and R2 are each, independently methyl groups; and R3 and R4 are each, independently carboxypropyl groups.

20. A compound of the formula: 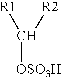
wherein R1 and R2 are each, independently, amino groups, and the compound has a high buffering capacity and a high conductivity in isoelectric state.

21. The compound of claim 20, wherein R1 and R2 are each, independently dimethylaminomethylene groups.

22. The compound of claim 20, wherein R1 and R2 are each, independently morpholinylmethylene groups.

23. The compound of claim 20, wherein R1 and R2 are each, independently dipropylaminomethylene groups.

24. The compound of claim 20, wherein R1 and R2 are each, independently piperidinylmethylene groups.

25. A method of making an ampholytic molecule comprising the steps of:
reacting a dialkylamine with an alkylester of a first halocarboxylic acid and an alkylester of a second halocarboxylic acid;
obtaining a quaternary ammonium compound bearing two exter groups in the reaction;
hydrolyzing the two ester groups of the quaternary ammonium compound forming an ampholytic compound; and
recovering the ampholytic compound in an isoelectric state, wherein the ampholytic molecule has a high buffering capacity and a high conductivity in isoelectric state.

26. The method of claim 25, wherein the dialkylamine is diethylamine and the alkylester of a first halocarboxylic acid and a second halocarboxylic acid is 4-bromobutanoic acid ethyl ester.

27. A method of making an ampholytic molecule comprising the steps of:
reacting a diaminoalcohol with an anhydride of a diprotic acid; and
recovering the ampholytic compound in an isoelectric state, wherein the ampholytic molecule has a high buffering capacity and a high conductivity in isoelectric state.

28. The method of claim 27, wherein the diaminoalcohol is 1,3-bis(N,N-dimethylamino)-2-propanol and the anhydride is a sulfur trioxide.pyridine complex.

29. A method of making an ampholytic molecule comprising the steps of:
reacting a secondary amine with an epihalohydrin;
obtaining a diaminoalcohol product from the reaction;
reacting the diaminoalcohol with an anhydride of a diprotic acid; and
recovering the ampholytic compound in an isoelectric state, wherein the ampholytic molecule has a high buffering capacity and a high conductivity in isoelectric state.

30. The method of claim 29, wherein the secondary amine is morpholine and the epihalohydrin is epichlorohydrin and the anhydride is sulfur trioxide.pyridine complex.

31. The method of claim 29, wherein the secondary amine is dipropylamine and the epihalohydrin is epichlorohydrin and the anhydride is sulfur trioxide.pyridine complex.

32. The method of claim 29, wherein the secondary amine is piperidine and the epihalohydrin is epichlorohydrin and the anhydride is sulfur trioxide.pyridine complex.

33. An ampholytic molecule comprising:
a first weakly acidic functional group having a first pKa;
a second weakly acidic functional group having a second pKa; and
a weakly basic functional group, wherein the ampholytic molecule has a unique pI value between the first pKa value and the second pKa value.

34. The molecule of claim 33, wherein an absolute value of a difference between the first pKa value and the second pKa values is less than three.

35. The molecule of claim 33, wherein the charge-carrying or chargable atoms of the first weakly acidic functional group, the second weakly acidic functional group, and the weakly basic functional group are individually separated by four or more covalent bonds.

36. An ampholytic molecule comprising one or more weakly acidic functional groups each having a pKa value and one or more weakly basic functional groups each having a conjugate acid form with a pKa value, wherein the ampholytic molecule has a unique pI value.

37. The molecule of claim 36, wherein an absolute value of a difference between a pKa value adjacent to and lower than the pI value and a pKa value adjacent to and higher than the pI value is less than three.

38. The molecule of claim 36, wherein four or more bonds separate the charge-carrying or chargable atoms of a first functional group and a second functional group having pKa values adjacent to the pI value.

39. A method of making an ampholytic compound comprising the steps of:
forming an ester-group bearing tertiary amine by reacting a secondary amine with a first ester;
reacting the ester-group bearing tertiary amine with a second ester to form a quaternary ammonium compound bearing two ester groups; and
forming an ampholytic compound by hydrolyzing the first ester group and the second ester group of the quaternary ammonium compound bearing the two ester groups, wherein four or more bonds separate the charge-carrying or chargable atoms of the first weakly acidic functional group and the second weakly acidic functional group and a nitrogen atom of the quaternary ammonium group.

40. The method of claim 39, wherein the first ester is formed from a first weak acid having a pKa value between 1 and 14 and a functional group capable of reacting with the secondary amine.

41. The method of claim 39, wherein the second ester is formed from a second weak acid having a pKa value between 1 and 14 and a functional group capable of reacting with the ester-group bearing tertiary amine.

42. The method of claim 39, wherein the ampholytic compound is formed by hydrolyzing the first ester group and the second ester group of the quaternary ammonium compound bearing two ester groups.

43. The method of claim 39, further comprising the step of purifying the ampholytic compound.
